(19) United States
(12) Patent Application Publication
(10) Pub. No.: US 2012/0094006 A1
(43) Pub. Date: Apr. 19, 2012

(54) Method Of Making A Skin Engaging Member

(76) Inventor: Michael Joseph Kwiecien, Scituate, MA (US)

(21) Appl. No.: 13/272,318

(22) Filed: Oct. 13, 2011

Related U.S. Application Data
(60) Provisional application No. 61/393,640, filed on Oct. 15, 2010.

(51) Int. Cl. B05D 1/36; A61K 8/84; A61Q 9/02; A61K 8/81

(52) U.S. Cl. 427/2.1; 424/73; 424/70.11; 424/70.15; 424/70.17; 424/70.12

(57) ABSTRACT

A method of forming a skin engaging member for use on a hair removal device comprising the steps of: providing a carrier having at least one receiving region; and dispensing a skin conditioning composition in a flowable form through a dispensing member, into or onto the receiving region, wherein said skin conditioning composition is dispensed while at least one of said carrier and said dispensing member is moving relative to the other, wherein the skin conditioning composition comprises at least one of: a water soluble polymer, an emollient, and a mixture thereof.

### CROSS REFERENCE TO RELATED APPLICATIONS

This application claims the benefit of U.S. Provisional Application No. 61/393,640 filed Oct. 15, 2010.

### BACKGROUND OF THE INVENTION

The use of shaving aids on razor blades to provide lubrication benefits during the shave is known. Some shaving aids have been described such as in U.S. Pat. Nos. 7,121,754; 6,298,558; 5,711,076; 5,134,775; and U.S. Patent Publ. No. 2006/0225285 and 2008/060201. One example of a known shaving aid is the Hydro® “Gel Reservoir” by Schick which includes a reservoir positioned on a flip top back. Shaving aids are typically described for use between the razor blades and the rear cover cap. Recently, it has been reported that certain shaving aid materials which can be placed forward or aft of the razor blades. See U.S. Patent Publ. No. 2009/0223057. These shaving aid materials, however, require both a water-soluble shaving aid and a water-insoluble erodible medium wherein the water-soluble shaving aid is at least partially soluble or miscible with the water-insoluble erodible medium. Other razors having lubrication boxes with shaving aids have also been disclosed. See e.g. WO 2011/047221A; WO 2011/047222A; WO 2011/049892A; WO 2011/050140A.

Despite the numerous attempts to provide shaving aids onto razors, there remains a need for a hair removal device with a new skin conditioning system which can be formed in a simple continuous or batch process, wherein the skin conditioning system is capable of delivering various agents to skin during use which can be positioned on various portions of the handle to provide specific skin conditioning benefits.

### SUMMARY OF THE INVENTION

One aspect of the invention relates to a method of forming a skin engaging member for use on a hair removal device comprising the steps of: providing at least one carrier having at least one receiving region; and dispensing at least one skin conditioning composition in a flowable form through at least one dispensing member, into or onto the receiving region, wherein said skin conditioning composition is dispensed while at least one of said carrier and said dispensing member is moving relative to the other, wherein the skin conditioning composition comprises at least one of a water soluble polymer and optionally a water insoluble polymer; an emollient and an optional water insoluble structuring polymer; and a mixture thereof.

### BRIEF DESCRIPTION OF THE DRAWINGS

FIG. 1 is a side view of a hair removal device in accordance with at least one embodiment of the present invention. FIGS. 2 and 3 are cross sectional side views of additional hair removal cartridges in accordance with the present invention. FIGS. 4-9 are top planar views of various hair removal cartridges in accordance with one or more embodiments of the present invention. FIGS. 10-15 are side cross sectional views of various other skin engaging members in accordance with one or more embodiments of the present invention. FIG. 16 is a side view of an exemplary system for making a skin engaging member. FIGS. 17a and 17b depict views of dispensing members having multiple dispenses ports.

### DETAILED DESCRIPTION OF THE INVENTION

The hair removal device of the present invention comprises at least one skin engaging member which allows for the loading of various skin conditioning compositions onto the device for delivery during the hair removal process. The details of the skin engaging member and its location on the device will be disclosed herein with figures showing exemplary embodiments which can include various elements of the present invention. Those of skill in the art will understand that various combinations elements described in the specification and disclosed in the figures can be used in accordance with the present invention. This device is suitable for use during the hair removal process, such as shaving, and the skin conditioning composition(s) can include various known compounds commonly used for topical application in personal care.

The hair removal device generally comprises a hair removal cartridge, also commonly referred to as a “head”, and a handle or grip portion, upon which the hair removal cartridge is mounted. The hair removal device can be a manual or power driven, disposable or part of a system, and can be used for wet and/or dry application. The hair removal cartridge can include a wide scraping surface such as where the hair removal device is used with a depilatory, or a razor cartridge where the device is a shaving razor. The hair removal cartridge may be replaceable or pivotally connected to a cartridge connecting structure. In an aspect, the cartridge connecting structure includes at least one arm to releasably engage the hair removal cartridge.

### I. HAIR REMOVAL CARTRIDGE

The hair removal cartridge of the present invention comprises a hair removal member. The hair removal member is the structure responsible for cutting, pulling or shearing off the hair from the skin. In one embodiment, the hair removal member is one or more blades; in another embodiment, the hair removal member is a scraping edge which can be used after a depilatory is applied onto the skin to be treated. In yet another embodiment, the hair removal member comprises a plurality of tweezer member which can be used for epilation (pulling hairs out of the follicle).

The hair removal member can be one or more elongated edges, and/or blades (rotary, foil, or straight edged). Those of skill in the art will understand that the other types of hair removal member are also described in accordance with the present invention. In one embodiment, the hair removal member comprises one or more elongated edges positioned on the cartridge between a first and a second end, said one or more elongated edges comprising a tip extending towards said first end. For example, U.S. Pat. No. 7,168,173 generally describes a Fusion® razor that is commercially available from The Gillette Company. A variety of razor cartridges can be used in accordance with the present invention. Nonlimiting examples of suitable razor cartridges, with and without fins, guards, and/or shave aids, include those marketed by The Gillette Company under the Fusion®, Venus® product lines as well as those disclosed in U.S. Pat. Nos. 7,197,825, 6,449,849, 6,442,839, 6,301,785, 6,298,558; 6,161,288, and U.S. Patent Publ. 2008/060201. In one embodiment, where the elongated edge is a blade, the blade can comprise at least one inter blade guard as generally described in U.S. Pat. No. 7,681,314.

In one embodiment, the cartridge comprises a guard comprising at least one elongated flexible protrusions to engage a user's skin In one embodiment, at least one flexible protrusions comprises flexible fins generally parallel to said one or more elongated edges. In another embodiment, said at least one flexible protrusions comprises flexible fins comprises at least one portion which is not generally parallel to said one or more elongated edges. Non-limiting examples of suitable guards include those used in current razor blades and include those disclosed in U.S. Pat. Nos. 7,607,230 and 7,024,776; (disclosing elastomeric/flexible fin bars); 2008/0034590 (disclosing curved guard fins); 2009/0049695A1 (disclosing an elastomeric guard having guard forming at least one passage extending between an upper surface and a lower surface). In yet another embodiment, the hair removal device does not include a guard.

In one embodiment, at least one skin engaging member positioned on said cartridge can be forward or aft of said one or more elongated edges. The terms “forward” and “aft”, as used herein, define relative position between features of the cartridge (i.e., razor cartridge). A feature “forward” of the one or more elongated edges, for example, is positioned so that the surface to be treated with by the hair removal device encounters the feature before it encounters the elongated edges, for example, if the razor assembly is being stroked in its intended cutting direction, the guard is forward of the razor blades. A feature “aft” of the elongated edge is positioned so that the surface to be treated by the hair removal device encounters the feature after it encounters the elongated edges, for example if the razor assembly is stroked in its intended cutting direction, the cap is disposed aft of the razor blades.

Where a guard is present on the cartridge, the skin engaging member can be forward or aft of the guard. Where the skin engaging member is aft of the guard and forward of said elongated edge, the guard can remove any materials present on the skin, and allow any skin conditioning compositions in the skin engaging member to be released just ahead of the elongated edges.

### II. SKIN ENGAGING MEMBER

The skin engaging member comprises multiple components. In one embodiment, the skin engaging member comprises a carrier and at least one skin conditioning composition releasably engaged with said carrier. The carrier can be a flat or non-flat strip of material, a strip forming a trough, or even a reservoir substantially encasing the skin conditioning composition. “Releasably engaged” means that the skin conditioning composition is present on the carrier or at least partially contained in the carrier such that when the skin engaging member is exposed to water during use, the skin conditioning composition can be released by dissolution or dispensing out of the carrier.

Where the skin engaging member is in the form of a reservoir or sheath, the skin conditioning composition is deposited into the receiving region of the carrier, and an optional skin contacting member comprising at least one dispensing orifice can be placed onto said carrier to at least partially contain the skin conditioning composition within said carrier. Those of skill will understand that the carrier need not surround the skin conditioning composition completely. In another embodiment, the carrier forms an open trough, where the skin conditioning composition is present in the recess formed by the open trough.

In one embodiment, the skin engaging member forms at least one orifice for dispensing said skin conditioning composition onto skin during use. In one embodiment, the orifice has a cross sectional area of from about 0.0005 to about 0.25 square inches. Small orifices can also be provided with cross sectional area of from about 0.005 to about 0.05 square inches, or from about 0.01 to about 0.025 square inches. Larger orifices can have cross sectional areas of from about 0.05 to about 0.25 square inches, or from about 0.1 to about 0.2 square inches. Combinations of small and large orifices can also be provided on the same skin engaging member, or on separate skin engaging members on the same cartridge, depending on the desired dispense rate and amount of exposure of the skin conditioning composition to water.

In another embodiment, the skin engaging surface of the component has a surface area and said at least one orifice has a cross sectional area in a cross section area ratio of from about 50:1 to about 1:1. In another embodiment, the orifice has a greatest lateral distance of from about 1% to about 80% of the greatest lateral distance of the hair removal cartridge, or from about 2% to about 10%, or from about 3% to about 5%, or from about 15% to about 50%. Examples of suitable carriers include the sheaths disclosed in U.S. Pat. No. 6,298,558 or 7,581,318.

Where more than one skin engaging members is provided on the hair removal device, they can be the same or different. By different, meaning having a different carrier, a different skin conditioning composition, or wherein both carrier and skin conditioning composition are different. Of course, the hair removal device can include on skin engaging member of the present invention and a conventional lube strip or shaving aid. Where multiple skin engaging members are provided, the first and second skin engaging members can be formed of the same carrier. In such an embodiment, the carrier is used to form both the first and the second skin engaging members, the difference would be that the skin conditioning composition contained within the different portions of the carrier could be different. Where the same skin conditioning composition is contained throughout the carrier, the embodiment could be described as having a single skin engaging member where a first part of the skin engaging member is forward of said at least one elongated edges, and optionally a portion of the skin engaging member is positioned aft of said at least one elongated edges.

a. Skin Engaging Member Forming a Ring

In one embodiment, the skin engaging member is in the form of a straight line, positioned on the hair removal device such that it can contact skin during use. In another embodiment, the skin engaging member forms at least a partial ring around the majority of the outer periphery of the at least one elongated edge or least 75%, or up to 100% is surrounded by one or more carriers. In one embodiment, a single carrier is used to surround at least a portion of said one or more elongated edges, in another embodiment, multiple carriers and/or various compositions within the same carrier are used to surround at least a portion of said one or more elongated edges.

In one embodiment, the carrier or carriers can form a 270 degree ring around the entire perimeter of the at least one elongated edge, or even 360 degree ring. The carrier need not be in contact or be immediately adjacent to the elongated edge but by providing a ring around the elongated edge, the carrier is able to ensure that the skin conditioning composition is deposited onto skin prior to and immediately following any contact of the elongated edge to skin.

The carrier or carriers can have smooth curve sections as it forms the corners around the edges of the at least one elongated edge, or it can form sharp edges which are affixed onto one another via melt bonding or adhesives. Where a non-linear section (curved, angled, partially twisted, etc) of the carrier is needed, the skin conditioning composition can be formed integral with the carrier (formed at the same time), or they can be formed separately then later the skin conditioning composition can be transferred into the carrier. In one embodiment, the skin engaging member is formed as a linear article where the non-linear deformations are formed while the skin engaging member is still heated from the making or assembly process, after the non-linear portions are formed, the member can then be left to cool and harden. In yet another embodiment, the carrier is formed without any skin engaging composition contained therein, then non-linear portions are formed, or the carrier can be molded having non-linear portions. The skin conditioning composition can then be added into the area within the carrier. These and other ways of making the skin engaging member are also possible and will be described hereafter.

b. Optional additional shave aid or lube strip

In another embodiment, the hair removal device, further comprises a skin conditioning composition (such as a known or commercially available shave aid or lube strip) positioned intermediate said second end and said one or more elongated edges, said skin conditioning composition comprising a water insoluble polymer and a water soluble polymer. The optional shave aid can be positioned forward of aft of the elongated edges, preferably opposite the skin engaging member of the present invention. Non-limiting examples of known skin conditioning compositions suitable for use herein include shave aids and lubrication strips as described in: U.S. Pat. Nos. 7,069,658, 6,944,952, 6,594,904, 6,302,785, 6,182,365, D424,745, 6,185,822, 6,298,558 and 5,113,585, and 2009/0223057.

c. Carrier

The carrier can be formed of a variety of materials. In one embodiment, the carrier is non-water soluble such that it does not degrade or dissolve during normal use. In another embodiment, the carrier can be partially water-soluble to allow for release of the skin conditioning composition. The partially water-soluble carrier is preferably less soluble than the skin conditioning composition and will preferably wear more slowly. In one embodiment, the carrier can be made of a shave aid or lubrication strip material as described in the prior paragraph. Without intending to be bound by theory, it is believed that having a carrier which dissolves and/or wears more slowly than the skin conditioning composition will allow for increased exposure of the skin conditioning composition contained therein over time. Increasing exposure of the skin conditioning composition over time will allow for maintained and/or increased release of the skin conditioning composition even as it begins to wear down. This can be particularly desirable where the skin conditioning composition contained within the carrier.

The carrier should have sufficient mechanical strength and rigidity to provide adequate mechanical strength to the entire skin engaging member, both as initially produced and after a significant amount of water soluble material has been leached out of the skin engaging member. In one embodiment, the carrier comprises a base and one or more side walls, forming a receiving region onto or into which the shave conditioning composition is placed. In another embodiment, the carrier forms one or more retaining members within said receiving channel extending away from said base. In one embodiment, the retaining member is elongated and stretches across at least about 20% to about 100%, or from about 35% to about 75%, or about 50%, of the length of the carrier. In another embodiment, a plurality of retaining members are used, in a linear or non linear alignment throughout the receiving region. The retaining members can be space equidistantly or not. In yet another embodiment, one or more of the retaining member has a height of from about 0.05 cm to about 1 cm, or from about 0.1 cm to about 0.5 cm. In another embodiment, one or more of the retaining members has a height of from about 10% to about 100%, or from about 30% to about 60%, of the height of at least one said side walls.

In one embodiment, the side walls are not the same height (as measured extending away from the base of the carrier). At least one of side walls can have a height of about 0.1 cm to about 1 cm, preferably from about 0.2 cm to about 0.4 cm. The pair of side walls can be biased away from each other as the walls extend away from said base, or they can be biased towards each other. In yet another embodiment, one or both ends of the carrier can be enclosed as described in US 7,581,318.

In one embodiment, the carrier is made of a water-insoluble polymer, particularly a thermoplastic resin. Thermoplastic resins are those materials which can be extruded or molded into a shape and are resilient under normal environmental conditions such as contact with water, even up to normal household hot water temperatures (for example up to 125° C.); normal wear and tear by consumers during use; device assembly and shipping, etc. Thermoplastic resins suitable for use in the carrier include polystyrene, high impact polystyrene (polystyrene-butadiene), polypropylene, filled polypropylene, polyethylene, nylon ethylene vinyl acetate, and blends such as 70% nylon/30% polyethylene oxide, 60% polystyrene/40% polyethylene oxide butadiene styrene copolymer, polyacetal, acrylonitrile-butadiene styrene copolymer, and mixtures thereof. The preferred resins are high impact polystyrene, polystyrene, ethylene vinyl acetate (EVA), and mixtures thereof.

In one embodiment the carrier can be made of a solid polymeric composition similar to those materials described below in reference to shaving aids. In yet another embodiment, the carrier comprises a mixture of water-soluble and water-insoluble polymers similar to the shaving aids described below. Without intending to be bound by theory, this can be particularly desirable where it is desirable for the entire skin engaging member to be erodible or at least partially water soluble. In one embodiment, said water-insoluble polymer is present at a level of at least about 35% by weight of said carrier, or at least about 50%, or at least about 75%, or at least about 90%. The water-soluble polymer can be present as the remainder. Optionally, the carrier can include additives such as lubricants or plasticizers, fillers such as CaCO3, and colorants such as TiO2.

In one embodiment, the carrier can be made of a shave aid composition as described below. This can be particularly useful if the carrier is intended to be wearable and/or dissolvable throughout the usage life of the device and/or skin engaging member. Further, by providing a carrier made of a shave aid composition, both parts of the skin engaging member provide hair removal benefits during use.

In one embodiment, a wear indicating effect is produced when the carrier and the skin conditioning composition are made of disparately colored materials (e.g. white colored sheath and blue colored core). The skin conditioning composition leaches out of the skin engaging member through use. With sufficient use, a colored region within the skin conditioning composition leaches out. By examining the release holes along the skin engaging surface, the user is provided with an indication that the shaving unit and/or skin engaging surface have reached their effective life. In one embodiment, the skin conditioning composition consists of polyethylene oxide/polystyrene blend which is colored with Indigotine, FD&C #2 dye and the sheath consists of nylon and/or polystyrene which has been colored white.

Further, the carrier may be affixed to the cartridge by adhesive such as Loctite Super Bonder 499, by mechanical locking mechanism, by thermal welds or by a combination thereof.

d. Skin Conditioning Compositions

Skin conditioning composition, as referred to herein, means any suitable skin conditioning related composition which can be used for topical application onto skin so long as they can be deposited into or onto the carrier as described herein. The skin conditioning composition of the present invention can be a solid or a fluid as long as it stays on or within the skin engaging component prior to use and can be dispensed (such as out of said one or more orifices) during the hair removal process. In embodiments, where the composition is a fluid, the size of the orifice can be tailored to control the flow of composition out of the orifices depending on the flow viscosity of the composition and other compositional details which can be determined by one of ordinary skill in the art. Generally, the fluids such as water have a low viscosity and are considered “thin” whereas fluids such as honey have a higher viscosity and are considered “thick.” In embodiments where the composition has a low viscosity, such as from about 0.5 to about 2 Pa*s, the skin engaging member can include at least one small orifice, or only small orifices, as defined above. Where the composition has a higher viscosity, the skin engaging member can have an open top end, or include one or more large orifices, as defined above. Various combinations of small and/or large orifices can of course be used for solid skin conditioning composition executions.

i. Shaving Aids

Skin conditioning composition in the form of solid polymer cores are commonly used on commercially available razors and are commonly referred to as shaving aid. In one embodiment, the shaving aid is selected from the group consisting of polyethylene oxide, polyvinyl pyrrolidone, polyacrylamide, hydroxypropyl cellulose, polyvinyl imidazoline, polyethylene glycol, poly vinyl alcohol, polyhydroxyethylmethacrylate, silicone copolymers, sucrose stearate, vitamin E, soaps, surfactants, panthenol, aloe, plasticizers, such as polyethylene glycol; beard softeners; additional lubricants, such as silicone oil, Teflon® polytetrafluoroethylene powders (by DuPont), and waxes; essential oils such as menthol, camphor, eugenol, eucalyptol, safrol and methyl salicylate; tackifiers such as Hercules Regalrez 1094 and 1126; non-volatile cooling agents, inclusion complexes of skin-soothing agents with cyclodextrins; fragrances; antipruritic/counterirritant materials; antimicrobial/keratolytic materials such as Resorcinol; anti-inflammatory agents such as Candilla wax and glycyrrhetinic acid; astringents such as zinc sulfate; surfactants such as pluronic and iconol materials; compatibilizers such as styrene-b-EO copolymers; and combinations thereof. The shaving aid(s) may release from the surface to provide improved shaving. The shaving aid(s) can be dispersed throughout the skin conditioning composition and, additionally, the carrier may also contain small amounts of the shaving aid(s). In one embodiment, said shaving aid is a water soluble or water miscible material; however, non-water soluble additives can also be incorporated as long as they can be carried out with a water soluble core component.

In one embodiment, the skin conditioning composition forms a solid polymeric core comprises a water soluble polymer comprising polyethylene oxide, polyvinyl pyrrolidone, polyacrylamide, polyhydroxymethacrylate, polyvinyl imidazoline, polyethylene glycol, polyvinyl alcohol, polyhydroxyethymethacrylate, silicone polymers, or a mixtures thereof. The water-soluble polymer can be at a level of at least 50%, more preferably at least 60%, by weight of the skin conditioning composition, up to about 100%, or up to about 90%. Suitable polyethylene oxides include POLYOX (from Union Carbide) or ALKOX (from Meisei Chemical Works, Kyoto, Japan). These polyethylene oxides will preferably have molecular weights of about 100,000 to 6 million, most preferably about 300,000 to 5 million. Other suitable polyethylene oxides include blends of about 40 to 80% of polyethylene oxide having an average molecular weight of about 5 million (e.g. POLYOX COAGULANT) and about 60 to 20% of polyethylene oxide having an average molecular weight of about 300,000 (e.g. POLYOX WSR-N-750). The polyethylene oxide blend may also advantageously contain up to about 10% by weight of a low molecular weight (i.e. MW<10,000) polyethylene glycol such as PEG-100.

In one embodiment, the shaving aid further comprises a polycaprolactone at a level of from about 0.5% to about 50%, or from about 1% to about 20%, or from about 1% to about 10%, by weight of the shaving aid. The polycaprolactone can have a number average mol weight of from about 1,000 to about 80,000 Daltons, or from about 30,000 to about 60,000 Daltons, or about 50,000 Daltons. Suitable polycaprolactones include polycaprolactone homopolymers as described in U.S. Pat. No. 6,302,785 at col. 4, lines 11-37. In another embodiment, at least a portion of the water-soluble polymer forms a discrete portion of the shaving aid. In yet another embodiment, part or all of the water-soluble polymer portion is coated with mineral oil as described in U.S. Patent Pub. No. 2008/0060201 at paragraph 23.

The shaving aid may also advantageously comprise a non-volatile cooling agent or an inclusion complex of a skin-soothing agent with a cyclodextrin, preferably in amounts up to about 25%, most preferably 10 to 20%, by weight of the skin conditioning composition. By non-volatile cooling agent is meant an agent which has a physiological cooling effect on the skin and which is appreciably less volatile than menthol. Preferably, the nonvolatile cooling agent will be one which when subjected to thermogravimetric analysis (e.g. using a 951 Thermogravimetric Analyzer from Dupont with a 20° C. temperature rise—̂ per minute) will retain at least 50% of its initial weight at a temperature of 160° C., more preferably at least 80% of its initial weight at 160° C., and most preferably at least 50% of its initial weight at 175° C.

Suitable cooling agents which can be utilized include non-volatile menthol analogs such as menthyl lactate, menthyl ethoxyacetate, menthone glycerinacetal, 3-1menthoxypropane-1,2-diol, ethyl 1-menthyl carbonate, (IS, 3S ,4R)-p-menth-8-en-3-ol, menthyl pyrrolidone 25 carboxylate, N-substituted-p-menthane-3-carboxamides (as described in U.S. Pat. No. 4,136,163, which is incorporated herein by reference) including, for example, N-ethyl-pmenthane-3-carboxamide, acyclic carboxamides

Suitable skin-soothing agents which can be utilized in the cyclodextrin inclusion complex include menthol, camphor, eugenol, eucalyptol, safrol, methyl salicylate, and the aforedescribed menthol analogs. Any suitable cyclodextrin may be utilized to form the inclusion complex including alphacyclodextrin, beta-cyclodextrin, gamma-cyclodextrin and modified cyclodextrins such as hydroxypropyl-beta-cyclodextrin, methyl-beta-cyclodextrin., and acetyl-betacyclodextrin. The preferred cyclodextrins are betacyclodextrin and gamma-cyclodextrin.

When the shaving aid comprises a cyclodextrin inclusion complex, the skin conditioning composition may also advantageously comprise up to 65 about 10%, preferably about 2 to 7%, by weight of a displacing agent which displaces the skin-soothing agent from the inclusion complex upon contact with water, thereby enhancing the release of the skin-soothing agent from the skin conditioning composition material during use. The displacing agent is a material which is capable of forming a more stable complex with the cyclodextrin than the complex formed with the skinsoothing agent and, thus, displaces the skin-soothing agent from the complex when the shaving aid is contacted with water. Suitable displacing agents include surfactants, benzoic acids, and certain amines (e.g. urea). Further details with respect to the aforementioned cooling agents, cyclodextrin inclusion complexes and displacing agents may be found in U.S. Pat. Nos. 5,653,971, and, 5,713,131.

ii. Emollients

In another embodiment, the skin conditioning composition, at least partially contained within said carrier, comprises at least one emollient. In one embodiment the emollient is hydrophobic. As explained above, the skin conditioning composition can be a solid or can be fluidic. In certain embodiments, the composition can consist essentially of one or more emollients which could form a fluid at 25° C. In such embodiments, depending on the viscosity of the composition, varying orifice sizes can be used to control the dispensing of emollient during use.

The emollient is liquid, semi-solid and/or solid at room temperature. In one embodiment the emollient may comprise one or more hydrocarbon emollients, a lipid, lipophilic skin conditioning actives, or a mixture thereof. Suitable lipids include fatty acyls such as fatty acids, fatty alcohols, esters, triglycerides, fats, butters, and waxes; glycerolipids; glycerophospholipids; sphingolipids; sterol lipids; prenol lipids; saccharolipids; polyketides; lipophilic skin active agent emollients, and mixtures thereof. Details on various suitable liquid, semi-solid and/or solid emollients are provided below.

Hydrocarbon emollients include straight chain, branched chain, saturated and unsaturated hydrocarbons and mixtures thereof and they may comprise natural or synthetic hydrocarbon emollients and mixtures thereof. Preferred natural hydrocarbon emollients include petrolatum, mineral oil and mixtures thereof. Preferred synthetic hydrocarbon emollients include branched chain hydrocarbons, such as isohexadecane (such as Arlamol HD™ from Croda) and Polydecene (such as Puresyn 2™ from Exxon Mobil).

Fatty alcohol or fatty acid emollients include saturated and unsaturated higher alcohols, especially C₁₂-C₃₀ fatty alcohols and fatty acids, especially lauric, myristic, palmitic, stearic, arachidic or behenic. Ester emollients include esters of a C₁₂-C₃₀ alcohol and mixtures thereof, especially isopropyl myristate, isopropyl isostearate and mixtures thereof. Triglyceride emollients include synthetic or natural triglycerides, especially natural triglycerides derived from sunflower, avocado, olive, castor, coconut, cocoa and mixtures thereof. More preferred are coconut-derived triglycerides, such as the commercially available materials Myritol™ 312 and 318 (Cognis), Estasan™ (Croda) and Miglyol™ (Sasol). Fat and butter emollients include coconut butter, shea butter and mixtures thereof. Wax emollients include paraffin wax, microcrystalline wax, candellila, ozokerite and mixtures thereof. Preferably the emollient comprises paraffin wax. Advantageously, hydrophobic phase comprises some wax because waxes may bestow further improved hardness and erodability to the solid moisturising composition. Preferably, the erodible, solid moisturizing composition comprises from 2% to 20% and more preferably from 3% to 15% wax by weight of the erodible, solid moisturizing composition.

Another class of suitable lipids include lipophilic skin active agent emollients which include oil soluble vitamins, such as vitamin E derivatives, including vitamin E acetate and tocopherol nicotinate; oil-soluble vitamin A derivatives, such as retinyl palmitate, lanolin, ceramides, sterols and sterol esters, salicylic acid, camphor, eucalyptol and essential oils.

In one embodiment, the skin conditioning compositions comprises at least one emollient and a water insoluble structuring polymer forming an erodible, solid moisturizing composition. Examples of such compositions have been described as an erodible, solid moisturizing composition described in U.S. Non-Provisional application Ser. No. 13/026556, which claims priority to U.S. Application Ser. No. 61/305682 titled “HAIR REMOVAL DEVICE COMPRISING ERODIBLE MOISTURIZER” and U.S Non-Provisional application Ser. No. 13/026575, which claims priority to U.S. Application Ser. No. 61/305687 titled “HAIR REMOVAL DEVICE COMPRISING AN ERODIBLE MOISTURIZER”, both to Stephens et al.

As used herein, the term “solid” when used in relation to the erodible, solid moisturizing composition refers to compositions which are solid at 25° C. As used herein, the term “water-insoluble” when used in relation to the structuring polymer, means “very slightly soluble”, according to the United States' Pharmacopeia (USP) definition in 31/NF 26 Vol. 2 General Notices, Page Xvii., or less than “very slightly soluble”, which, using the USP definition, means that more than 1000 parts of solvent (water, in this case) are needed to dissolve 1 part of solute (the structuring polymer, in this case) at Standard Temperature and Pressure. As used herein, the term “soluble in” when describing the ability of the water-insoluble structuring polymer to dissolve in the hydrophobic phase means “soluble”, according to the US' Pharmacopeia definition in 31/NF 26 Vol. 2 General Notices, Page Xvii., or less than “soluble”, which, using the USP definition, means that less than 30 parts of solvent (the hydrophobic phase, in this case) are needed to dissolve 1 part of solute (the structuring polymer, in this case) at the melting point of the water-insoluble structuring polymer.

In one embodiment, the skin conditioning composition is an erodible, solid moisturizing composition has a Chatillon Hardness at 25° C. of about 0.50 kg to about 3.25 kg, preferably about 0.75 kg to about 3.00 kg, more preferably about 1.00 kg to about 2.50 kg, measured according to the protocol provided hereinbelow. It is believed that a skin conditioning composition having such Chatillon hardness provides beneficial rates of wear.

Any water-insoluble structuring polymer comprised within the erodible, solid moisturizing composition may be any water-insoluble structuring polymer which bestows appropriate wear properties to the erodible, solid moisturizing composition and is preferably a water-insoluble structuring polymer which may bestow a Chatillon Hardness in the above-defined ranges to the erodible, solid moisturizing composition. The structuring polymer is water-insoluble to assist miscibility with or solubility in the hydrophobic phase (at the melting point of the water-insoluble structuring polymer), which in turn may ensure a homogenous distribution of hydrophobic phase throughout the polymer and thus more even wear properties. In addition, the water soluble nature of the polymer may improve the durability of the polymer (and therefore also the erodible, solid moisturizing composition) versus more hydrophilic polymers which may solubilise and wash away during hair removal processes that employ water, such as wet shaving.

In one embodiment, the erodible, solid moisturizing composition comprises from 2% to 50%, preferably from 3% to 40%, more preferably 4% to 12% of water-insoluble structuring polymer by weight of the erodible, solid moisturizing composition. In one embodiment, the water-insoluble structuring polymer comprises a block copolymer. More advantageously, the block copolymer comprises a di-block copolymer, a tri-block copolymer, a multi-block copolymer, a radial block copolymer, a random block copolymer, or a mixture of these polymers. More advantageously still, the block copolymer comprises a tri-block copolymer.

In another embodiment, the block copolymer is a tri-block copolymer, then the tri-block copolymer preferably comprises a linear ABA tri-block polymer. Without wishing to be bound by theory, applicants believe that the A blocks aggregate creating domains, within which the hydrophobic phase may accumulate, connected together by the B-blocks. This structure may provide an appropriate hardness to bestow the requisite wear properties to the erodible, solid moisturizing composition, while also being flexible enough to be processed and not to crack or break during processing and/or use. Advantageously, the linear ABA block copolymer comprises styrene-butadiene-styrene (SBS) block copolymer, styrene-isoprene-styrene (SIS) block copolymer, styrene-ethylenebutylene-styrene (S-EB-S) block copolymer, or mixtures thereof. More advantageously, the linear ABA block copolymer preferably comprises styrene-ethylenebutylene-styrene (S-EB-S) block copolymer. More advantageously still, the weight ratio of styrene to butadiene in the S-EB-S is in the range 20:80 to 40:60 and preferably around 30:70. Particularly useful commercially available ABA block copolymers include Versagel™ materials available from Penreco and the Kraton™ G series, especially G-6150, G-1651, G-1652 and 1654.

In yet another embodiment, the structuring polymer comprises a random block copolymer. An example of a suitable random block copolymer is ethylene vinyl acetate (EVA) which is a copolymer of ethylene and vinyl acetate. Advantageously, the amount of ethylene comprised within the EVA polymer is from 65-90%, preferably from 70-85% by weight of the EVA to give beneficial wear properties. A commercially available range of EVA is called Elvax™, by DuPont.

According to the invention, the erodible, solid moisturizing composition comprises at least about 50% hydrophobic phase by weight of the erodible, solid moisturizing composition. Preferably, the erodible, solid moisturizing composition comprises from 60% to 95% and more preferably from 70% to 90% hydrophobic phase by weight of the erodible, solid moisturising composition.

The erodible, solid moisturizing composition may comprise one or more additional components which bestow a suitable melt viscosity to the composition, such as oil phase gellants, to facilitate improved processing, provided that the additional component(s) do not significantly reduce the hardness or erodability of the erodible, solid moisturizing composition. Examples of such components are trihydroxystearin, which is commercially available as Thixcin R™ (manufactured by Elementis Specialities), ethylene vinyl acetate (EVA) and mixtures thereof.

Chatillon Hardness Test Method

Equipment: Chatillon TCD 200 equipped with a digital force gauge Sample preparation:
Step 1—Fully melt and cast lipid into 60 ml weigh boat (70 mm×70 mm×24 mm)
Step 2—Store lipid at 25° C. overnight to equibrilate. Step 3—Carefully remove lipid from weigh boat prior to hardness testing

A) Machine Preparation:
Step 1—Prepare Chatillon TCD 200 and digital force gauge according to manufacturers instructions. Step 2—Set the ramp speed to 47 mm/min.

B) Measuring the hardness value at 25° C.:
Step 1—The pointed geometry should be attached to the shaft of ramp for this test method. Step 2—Place the lipid sample as prepared above and on its side onto the metal base plate directly below the centre of the shaft of the ramp. The mid-point of the lipid should be in line with the centre of the shaft of the ramp. Step 3—With the lipid in place below the flat plate the speed set at 47 mm/min and the digital force gauge set at “C Peak” as above, depress the “Down” button on the Chatillon TCD200. Step 4—Stop the Chatillon TCD200 just as the probe touches the surface of the lipid and set the distance counter to zero. Step 5—Reset the force gauge so that it reads zero. Step 6—Depress the “Down” button on the Chatillon TCD200 until the distance counter reads 13 mm, record C Peak reading.

Melt Flow Viscosity Test Method

Melt flow viscosity is measured using a Brookfield DVII+ operating with RV spindles and various speeds as set forth below:
1. Positioning and leveling—Ensure that the viscometer is leveled perpendicular to the bench-top surface. Center the bubble level indicator.
2. Turn the viscometer on, then Autozero—Before readings may be taken, the viscometer must be autozeroed. This action is performed each time the power switch is turned on. Select the Spindle, Set the Speed.
3. Measurement
A.) Place a 250 g of sample into a 500 ml metal container and heat to between 110° C. until entire sample is molten. Place container into receiving area.
B.) Press yellow SELECT DISPLAY button until display changes from Deflection (%) to Viscosity (cP) for centipoise. (Note; SS=shear stress, SR=shear rate).
C.) Lower spindle vertically so that it is level with the level mark on the RV spindle.
D.) Measurements are taken by Singe Point Mode:
   1.) Press the red MOTOR ON/OFF button and watch the screen to ensure a cP reading is displayed. Record the reading after no less that 1 minute, or when reading is equilibrated and steady state shearing has been reached.
   2.) Press the SELECT DISPLAY button and watch the screen to ensure the Deflection reading is displayed. Record the reading after no less that 1 minute, or when reading is equilibrated and steady state shearing has been reached.

In one embodiment where the skin conditioning composition is an erodible, solid moisturizing composition made in accordance with Example 1, below, the skin conditioning composition has a melt viscosity as measured using an RV spindle 2 @ 100 rpm of from about 70 cps to about 160 cps at 110° C. The skin conditioning composition can also have a melt viscosity as measured using an RV spindle 2 @ 100 rpm of from about 160 cps to about 180 cps at 100° C. In another embodiment the skin conditioning composition has a melt viscosity as measured using an RV spindle 4 @ 100 rpm of from about 180 cps to about 1800 cps at 100° C. These melt flow viscosity measurements are determined by the MELT FLOW VISCOSITY Method defined below. Without intending to be bound by theory, it is believed that skin conditioning compositions having such melt viscosity are particularly suitable for slot coating or nozzle dispensing onto the carrier when forming the skin engaging member.

### III. DETAILS ON FIGURES

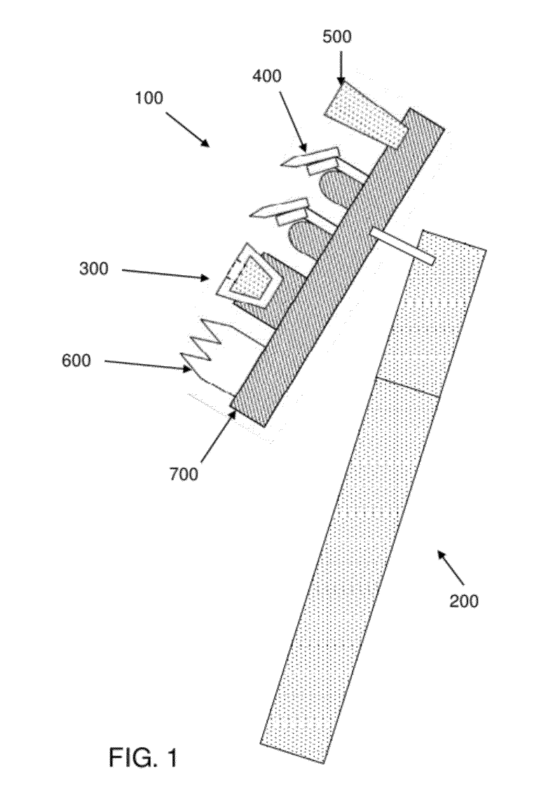

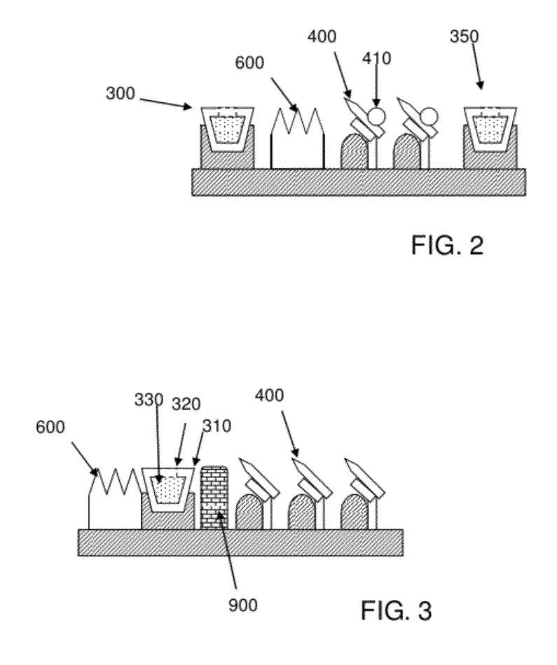

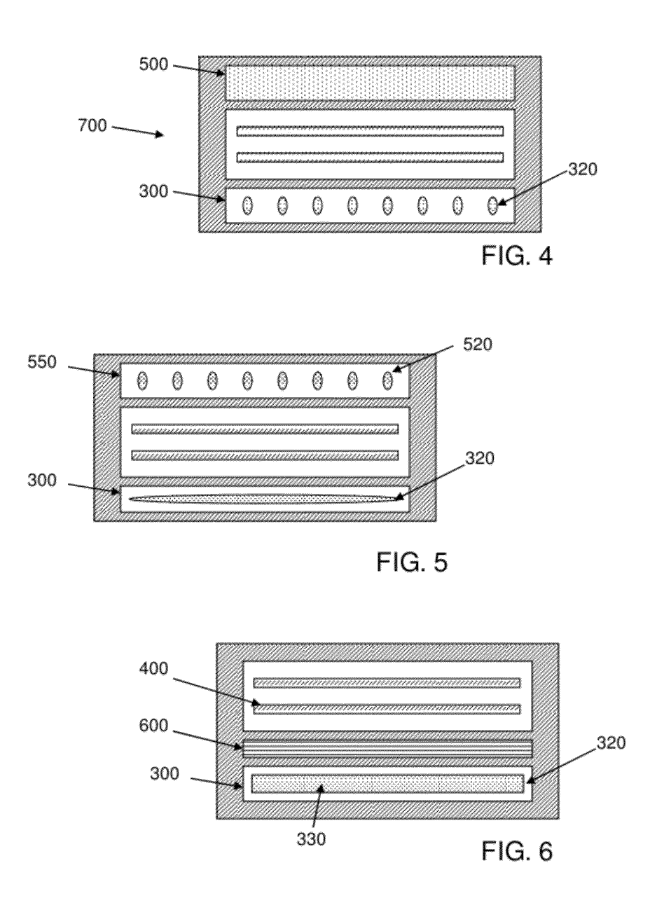

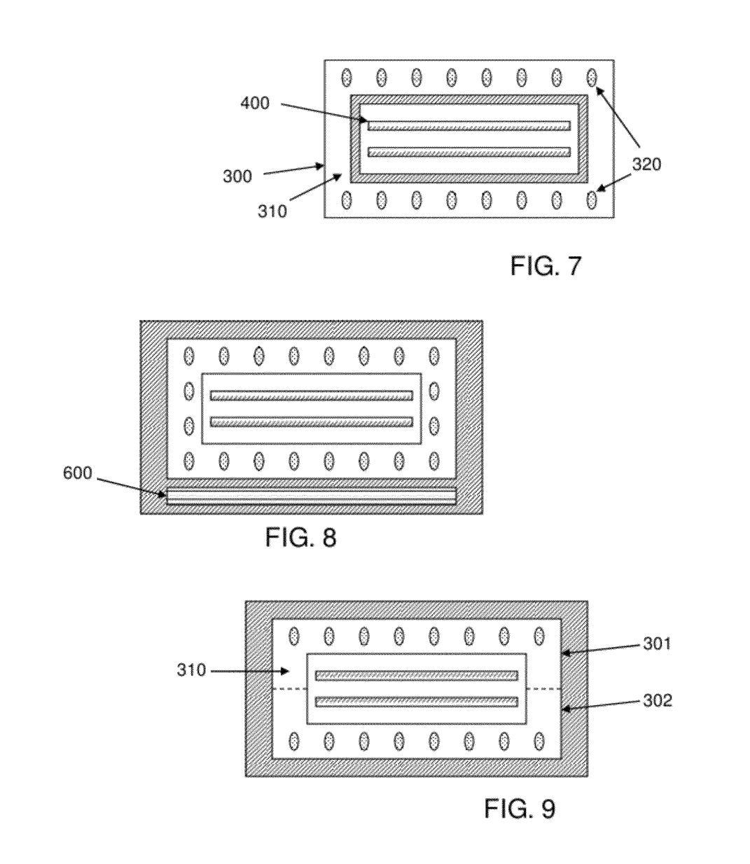

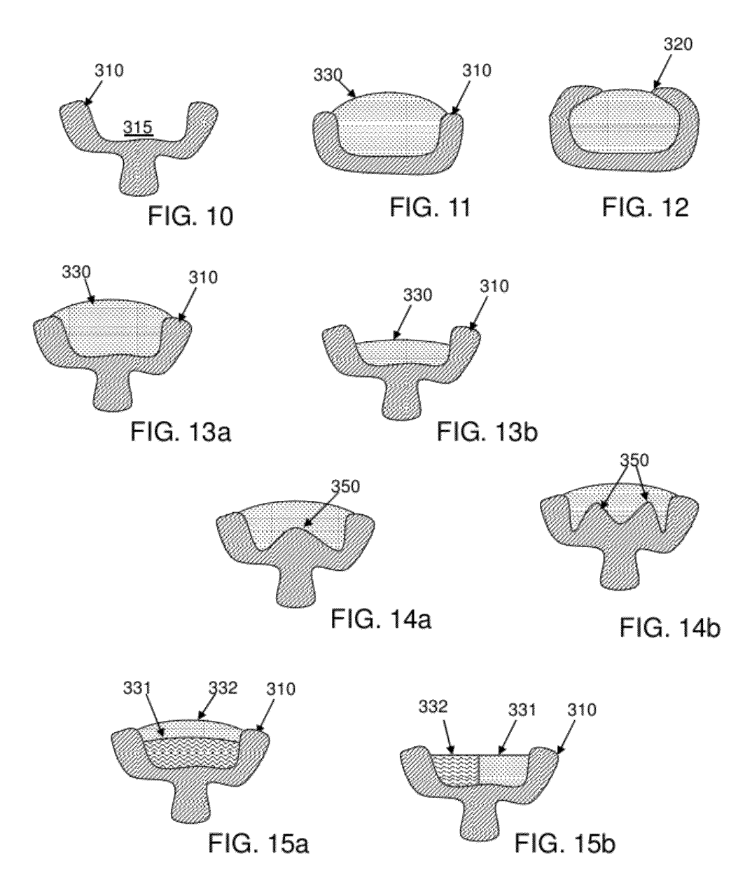

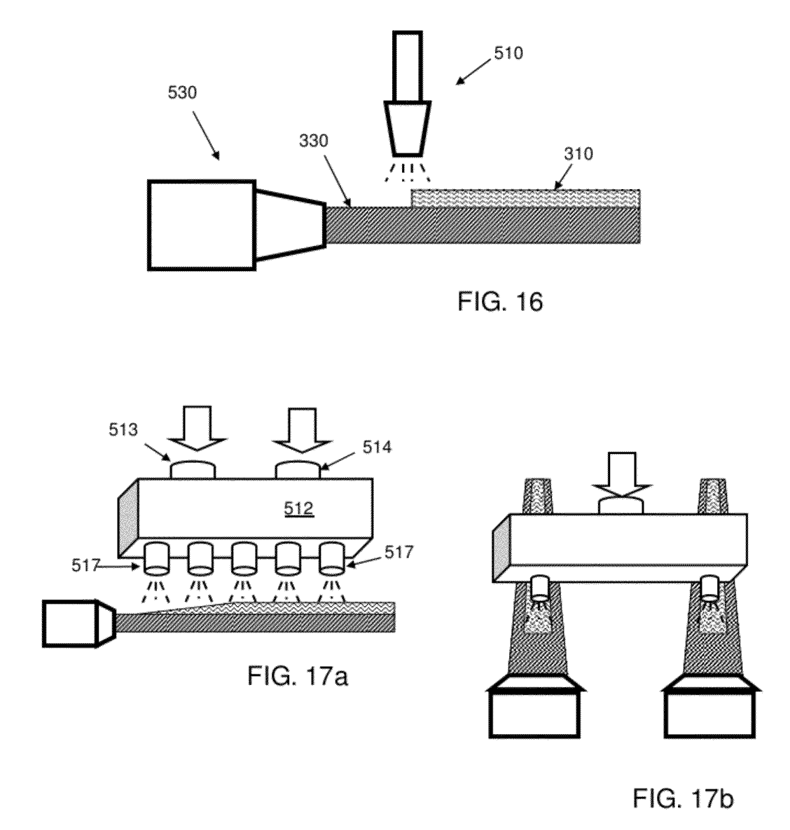

FIG. 1 is a side view of a hair removal device (100) in accordance with at least one embodiment of the present invention. The device comprises a hair removal cartridge (700) having a first end (710) and a second end (720). In this example the hair removal cartridge includes two elongated edges (400) and a skin engaging member (300) positioned forward of said two elongated edges. Further, a guard (400) is provided forward of said skin engaging member. A shave aid (500) is provided aft of said elongated edges.

FIG. 2 is a cross sectional side view of another hair removal cartridge in accordance with at least one embodiment of the present invention. Two elongated edges (400) are shown, which can be razor blades, having intrablade guards (410). In this example the skin engaging member (300) is positioned forward of elongated edges and forward of the guard, wherein the guard is positioned between the elongated edges and the skin engaging member. Further shown here is a second skin engaging member (350), positioned aft of the elongated edges. Those of skill in the art will understand that a hair removal device in accordance with the present invention can include any known on board chemistry (shave aid, lubrication strip, or skin engaging member) positioned aft of the elongated edge.

FIG. 3 is an embodiment where more than two elongated edges are provided on the hair removal cartridge, in one case three, four, or five elongated edges can be included. Also shown in this figure is the carrier (310) forming an orifice (320) to allow the skin conditioning composition (330) to be dispensed during use onto skin, and a structure (900) forward of the leading blade which can form a fixed position with respect to where the skin will pass prior to being contacted with the blades. Those of skill in the art will understand that where a shaving aid is positioned forward of the blades, it can be preferable for the shaving aid or another structure of the cartridge housing to form a fixed position with respect to the immediately following blade tip. The structure forming the fixed position can be formed integral to the housing or can be a separate component affixed to the housing. Having a fixed position can help ensure skin travels over the cartridge in a controlled orientation. In one embodiment, where the shaving aid positioned forward of the blades, the shaving aid can comprise a carrier which will not erode or otherwise deform during use.

FIG. 4 is a top planar view of a hair removal cartridge (700) in accordance with at least one embodiment of the present invention, wherein multiple orifices (320) are formed in the carrier. FIG. 5 is a top planar view of a hair removal cartridge where the skin engaging member forward of the elongated edges comprises at least one orifice having a greatest lateral distance which is at least a 50% of the greatest lateral distance of the hair removal cartridge. This embodiment also shows a second skin engaging member positioned aft of the elongated edges. FIG. 6 is a top planar view of a hair removal cartridge in accordance with at least one embodiment of the present invention, wherein a guard (600) is provided but not positioned forward of the skin engaging member. In another embodiment, an alternative or additional guard is positioned forward of the skin engaging member.

FIGS. 7 and 8 show top planar views of two hair removal cartridge in accordance with the present invention, wherein the skin engaging member at least partially encircles the elongated edges. Here the skin engaging member is shown forming a 360 ring around the elongated edges but a partial ring can also be within the scope of the present invention. In FIG. 7, the orifices do not extend around the periphery of the hair removal cartridge, whereas the orifices in FIG. 8 do. In one embodiment, the orifices are equally space apart. The hair removal cartridge of FIG. 8 also shows an embodiment where a guard (600) with fins is provided forward of the skin engaging member and hair removal elements. FIG. 9 shows an embodiment, where two skin engaging members are provided wherein they can be formed of the same carrier, or separate carriers which appear to be connected when viewed from a top view. The skin conditioning compositions can be the same or they can be different and tailored to deliver various skin conditioning compositions based on their position on the hair removal cartridge.

FIGS. 10-15b are various side cross sectional views of skin engaging members in accordance with the present invention. FIG. 10 shows the carrier (310) forming a receiving region (315) into which skin conditioning composition can be placed. FIGS. 11 and 12 show two additional side cross sectional view of skin engaging members where the side walls of the carrier can be shaped to form an orifice (320). FIGS. 13a and 13b show the same carrier (310) where the skin conditioning composition (330) has been worn down from use. FIGS. 14a and 14b show two skin engaging members where the carrier forms at least one retaining member (350) extending away from the base of the carrier. FIGS. 15a and 15b show two skin engaging members with a first skin conditioning composition (331) and a second skin conditioning composition (332) forming discrete layers within the carrier (310). The layers can be adjacent or they can be separated by a retaining member if so desired. The layered skin conditioning compositions shown in FIG. 15a can be created by repeated slot coating applications, or by separately molding then forming the layers and allowing them to be compressed or heated to mold together. The layers can also be made by a mixture of slot coating the first skin conditioning composition then molding the second skin conditioning composition thereon. Where the skin conditioning compositions are form side by side layers as in FIG. 15b, the layers can be formed by co-extrusion via a two orifice die, or they can be formed by having a two orifice die used on a slot coater. Where multiple layers of skin conditioning compositions are used, they can be similar with one or more ingredients, such as a fragrance or colorant or a skin conditioning active such as menthol, being changed. Further, more substantial differences between the first and second layer can also be used. For example, the first layer can be an emollient and structuring polymer and the second layer can be a water soluble polymer with a water insoluble polymer.

FIG. 16 is a side view of an exemplary system for making a skin engaging member comprising an extruder (530) forming a carrier (330) and a slot coating spray nozzle (510) forming a skin conditioning composition (310) on or about said carrier.

FIG. 17a depicts a dispensing member (512) having multiple dispenses ports (517). This dispense member comprises two feeds (513) and (514). The feeds can be mixed within dispenses member (512) or they can be dispensed onto the carrier separately to form multiple layers or in varying sequences to allow for selective dispensing of different skin conditioning compositions. In this embodiment, the carrier travels along the path of multiple dispense ports. This can be particularly desirable where multiple layers of the same or different skin conditioning compositions is desired.

FIG. 17b depicts a dispensing member where multiple ports are aligned perpendicular to the direction of the carrier. In this embodiment, different carriers can be treated with the same skin conditioning composition. Those of skill in the art will understand that multiple feeds can also be used similar to the embodiment shown in FIG. 17a. Those of skill in the art will understand that multiple dispensing members can also be used in series to allow for multiple layering or to facilitate change over of skin conditioning compositions with minimal changes to the manufacturing process. As explained above, the dispensing members can have one or more dispensing ports.

### IV. METHODS OF MAKING

The skin engaging member can be made by a variety of ways. Non-limiting examples of suitable methods of making the skin engaging member include: slot coating the skin conditioning composition into or onto the carrier, dual extrusion of the carrier and the skin conditioning composition, separately molding and/or extruding the carrier and skin conditioning composition then later assembly, and so forth. Non-limiting examples of ways to coextrude, or separately mold/extrude then assemble are provided in U.S. Pat. Nos. 7,121,754 and 6,298,558.

In one embodiment, the method comprises the steps of: providing a carrier having at least one receiving region; and dispensing a skin conditioning composition in a flowable form through a dispensing member, into or onto the receiving region, wherein said skin conditioning composition is dispensed while at least one of said carrier and said dispensing member is moving relative to the other, wherein the skin conditioning composition is can be selected from the group consisting of: a water soluble polymer and a water insoluble polymer, an emollient and a water insoluble structuring polymer. The receiving region can be the exterior of the carrier, or an inner volume defined by the carrier. In one embodiment, the skin conditioning composition is formed by dispensing a fluid skin conditioning composition into or onto the receiving region of the channel. The fluid skin conditioning composition can be in liquid phase, a flowable powder, a semi-solid phase, and so forth. In one embodiment, said skin conditioning composition would be solid at 25° C. and atmospheric pressure. The skin conditioning composition can be heated to at least the melting point of said skin conditioning composition to form a liquid during the dispensing step. The fluid is allowed to harden if a solid or semi-solid skin conditioning composition is desired.

If the fluid is a flowable powder, the powder can be sintered. In another embodiment, the skin conditioning composition is separately formed (such as by extrusion) then placed into or onto the receiving region and pressed or molded to fit.

In another embodiment, the skin conditioning composition can be solid or semi-solid prior to being applied to the receiving region, for example, it can be extruded or molded in a specific shape, then placed into or onto the receiving region. The skin conditioning composition can then be heated or pressurized to allow it to fit within the receiving region, then allowed to cool and/or solidify. In one embodiment, the step of dispensing said skin conditioning composition can be done by at least partially coating said skin conditioning composition onto a strip formed on said carrier.

In one embodiment, the skin conditioning composition is dispensed in a fluid form via using a mechanical pump such as a gear pump or a positive displacement piston pump to transfer the fluid from a holding tank to the dispense member or members. The holding tank can be heated if the skin conditioning composition needs to be melted to form a fluidic state. In another embodiment, the skin conditioning composition stored in the holding tank can be transferred to the dispense member(s) via pressurized gas (i.e. air or nitrogen) applying pressure onto the composition in the holding tank to drive fluid towards and out of the dispense member(s).

In one embodiment, the carrier is moving during the dispensing step. In such an embodiment, the carrier could be fed towards the dispensing mechanism for the skin conditioning composition where the receiving region would be filled as the carrier passes. The carrier can move at a rate of from about 1 feet/min to about 100 feet/min, or from about 20 feet/min to about 80 feet/min, or from 50 feet/min to about 70 feet/min In another embodiment, the carrier is provided and held stationary during the dispensing step and the skin conditioning composition is dispensed into the stationary carrier by way of a moving dispensing member (i.e. a moving nozzle or sprayer head). One example of this can be where the carrier is formed then placed on trays where the carrier is oriented in a perpendicular position. The trays can be fed along a line, where the dispensing members dispense into each carrier while moving along the perpendicular position of the carrier. Once one carrier is filled, the tray moves down exposing another carrier in a perpendicular orientation. In yet another embodiment, the carrier and the dispensing member can be moving simultaneously, either towards or away from one another.

The skin conditioning composition can be dispensed at a rate of from about 1 mL/min to about 300 mL/min, or from about 10 mL/min to about 200 mL/min, or from about 80 mL/min to about 120 mL/min, or about 100 mL/min, depending on the amount of carrier being filled and the desired amount of skin conditioning composition to be filled therein or thereon.

In another embodiment, the process further comprises a step of separating said carrier into individual skin engaging members. This step can be done by chopping the carrier into individual pieces before or after the skin conditioning composition is dispensed. Where the separating step is done after the skin conditioning composition is dispensed it can be preferable, but not necessary, to allow the carrier and skin conditioning composition to rest and cool, such that the skin conditioning composition can at least partially solidify prior to separating.

In one embodiment, the process further comprises a step of forming said carrier which can be performed prior to or simultaneously with either the step of providing the carrier and/or at applying said skin conditioning composition into or onto the receiving region of said carrier. In one suitable embodiment, the step of forming said carrier is performed simultaneously while the skin conditioning composition is coated into or onto the receiving region. In another embodiment, the step of forming said carrier precedes said step of providing said carrier. In yet another embodiment, the step of providing said carrier comprises the step of forming said carrier. The process can be continuous where both the carrier and the skin conditioning composition are formed and combined continuously (such as where the carrier is extruded and where the skin conditioning composition is slot coated thereon).

The carrier can be formed from a variety of known methods. In one embodiment, the carrier is formed by molding or extrusion. Where the carrier is molded, the skin conditioning composition can be applied into or onto the receiving member after the carrier is formed or while the carrier is still taking its final shape. Where the carrier is formed by extrusion, the skin conditioning composition can be applied by slot coating into or onto the receiving region (by spray coating or nozzle coating), or the skin conditioning composition can be placed into or onto the receiving region, either during extrusion of the carrier or after extrusion.

Where the skin conditioning composition is slot coated into or onto the receiving region, a slot die coater can be used. In yet another embodiment, the carrier and skin conditioning composition are co-extruded. Where the skin conditioning composition is formed using nozzle coating, a melt-pump and a dispensing nozzle can be used. Slot coating the skin conditioning composition into or onto the carrier can be done using a Dynatec Dynamelt equipped with a 40.51 Equity C. die applicator (nozzle assembly). The skin conditioning composition is slot coated onto the carrier which is extruded in a conventional shave aid extruding process as described in U.S. Pat. No. 6,442,839 at col. 5, lines 47 to col. 6, line 12. In one embodiment, the carrier is formed via an extruder line speed of about 60 feet/min, this rate can be from about 1 foot/min to about 200 feet/min, or from about 25 feet/min to about 100 feet/min. A two liter volume of the skin conditioning composition can be raised to a temperature of about 275° C. and pumped onto the extruded carrier at approximately 60% pump speed. The carrier with skin conditioning composition slot coated thereon can then be allowed to cool.

In one embodiment, the steps of providing the carrier and forming the skin conditioning composition are performed simultaneously, such as where the carrier is extruded while the skin conditioning composition is slot coated into or onto the receiving region. The process can be continuous or batch.

Where the carrier comprises separate parts (such as having a separate top or base which is later heat sealed or adhered to the rest of the carrier, the separate part can be put in place after the skin conditioning composition is provided into or onto the receiving region. In one embodiment, the carrier containing the skin conditioning composition is allowed to cool and sufficiently at least partially solidify prior to being placed onto the hair removal cartridge. In another embodiment, the skin engaging member is placed onto the hair removal cartridge prior to the cooling/hardening of any parts thereof.

In one embodiment, the receiving region of the carrier is filled with one or more skin conditioning compositions. If multiple skin conditioning compositions are used, varying colors can be used to indicate the product usage to the user. For example, the portion of the skin conditioning composition consumed first can be white or blue and the portion available for us thereafter can be red or yellow to inform the user that a new hair removal cartridge may be needed. In another embodiment, only a portion of the receiving region is filled with the one or more skin conditioning compositions.

In one embodiment, the step of dispensing said skin conditioning composition into or onto said receiving region is performed while at least a portion of said carrier is within 5° C. of its melting point. In one embodiment, the process further comprises a step of cooling said at least partially coated receiving region to form said skin conditioning composition. The method of the present invention further comprises a step of attaching said at least partially coated receiving region onto a razor cartridge.

In another embodiment, the method further comprises a step of dispensing a second layer of a skin conditioning composition adjacent to said skin conditioning composition present in said receiving region. In anther embodiment, the step of dispensing a second layer of a skin conditioning composition can comprise a step of slot coating said second layer of skin conditioning composition. The second layer of a skin conditioning composition can be adjacent to said first skin conditioning composition as shown in FIGS. 15a and 15b. The second layer is applied before or after the first layer has solidified. In a continuous process, the skin engaging member comprising the first skin conditioning composition is then fed into another slot coating process where the second layer is applied atop or along side the first layer.

### V. EXAMPLES

The skin conditioning composition is made in accordance with any of the examples provided below. In one embodiment, the carrier is selected from a thermoplastic material such as a high impact polystyrene, polystyrene, ethylene vinyl acetate, or a mixture thereof. In another embodiment, the carrier is made of a mixture of water soluble and water-insoluble polymers, such as polyethylene glycol and polystyrene. The carrier can be extruded or molded as needed.

On or within the receiving region of the carrier is a skin conditioning composition. By separating the carrier from the skin conditioning composition, certain skin benefit agents/actives which may be damaged from the carrier making process can still be incorporated into the skin engaging member without sacrificing on processing wear and/or compositional incompatibility with carrier materials. In one example, the skin conditioning composition is one of examples 1 or 2, or a mixture thereof, below. Suitable skin conditioning compositions can also include water soluble polymers, as disclosed above.

### Example 1

| Trade Name | INCI Name | % w/w |
|---|---|---|
| White soft paraffin | Petrolatum | 44.0 |
| Mineral oil | Paraffinum Liquidum | 44.0 |
| Kraton G1650E | Hydrogenated Styrene/Butadiene copolymer | 5.0 |
| Thixcin R | Trihydroxystearin | 2.0 |
| Paraffin Wax SP206 | Paraffin | 5.0 |

The composition of Example 1 is manufactured by heating the hydrocarbons and waxes to 130° C., then adding the linear ABA tri-block polymer (Kraton G1650E) and mixing well until polymer has fully dissolved. The mixture is then cooled to 90° C. and the Thixcin added, after which the mixture is poured into a mould and allowed to cool to room temperature. The Chatillon Hardness of the formulation of Example 1 is 1.7.

### Example 2

| Trade Name | INCI Name | % w/w |
|---|---|---|
| Fractionated coconut | Caprylic/Capric Triglyceride | 85.0 |
| oil | | |
| Paraffin Wax SP206 | Paraffin | 7.5 |
| Kraton GRP6935 | Hydrogenated Styrene/Butadiene copolymer | 5.0 |
| Synthetic Beeswax | Arachidyl Behenate | 2.5 |

The composition of Example 2 is manufactured by heating the hydrocarbons and waxes to 130° C., then adding the linear ABA tri-block polymer (Kraton GRP6935) and mixing well until polymer has fully dissolved. The mixture is then cooled to 90° C., after which the mixture is poured into a mould and allowed to cool to room temperature. The Chatillon Hardness of the formulation of Example 2 is 0.6.

It should be understood that every maximum numerical limitation given throughout this specification includes every lower numerical limitation, as if such lower numerical limitations were expressly written herein. Every minimum numerical limitation given throughout this specification includes every higher numerical limitation, as if such higher numerical limitations were expressly written herein. Every numerical range given throughout this specification includes every narrower numerical range that falls within such broader numerical range, as if such narrower numerical ranges were all expressly written herein.

All parts, ratios, and percentages herein, in the Specification, Examples, and Claims, are by weight and all numerical limits are used with the normal degree of accuracy afforded by the art, unless otherwise specified.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”

All documents cited in the DETAILED DESCRIPTION OF THE INVENTION are, in the relevant part, incorporated herein by reference; the citation of any document is not to be construed as an admission that it is prior art with respect to the present invention. To the extent that any meaning or definition of a term or in this written document conflicts with any meaning or definition in a document incorporated by reference, the meaning or definition assigned to the term in this written document shall govern. Except as otherwise noted, the articles “a,” “an,” and ^{the} _{mean one} or more.”

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A method of forming a skin engaging member for use on a hair removal device comprising the steps of:
a. providing a carrier having at least one receiving region; and
b. dispensing a skin conditioning composition in a flowable form through a dispensing member, into or onto the receiving region,
wherein said skin conditioning composition is dispensed while at least one of said carrier and said dispensing member is moving relative to the other.

2. The method of claim 1, wherein the carrier is moving during the dispensing step, preferably at a rate of from 1 feet/min to about 100 feet/min.

3. (canceled)

4. The method of claim 1, wherein said skin conditioning composition is dispensed at a rate of from about 1 mL/min to about 300 mL/min

5. The method of claim 2, further comprising a step of separating said carrier into individual skin engaging members.

6. The method of claim 5, further comprising a step of resting said carrier with the skin conditioning composition contained in or on the receiving region, wherein said step of resting occurs before said step of separating said carrier.

7. The method of claim 1, wherein said the carrier is stationary during the dispensing step and a dispensing member which dispenses the skin conditioning composition moves along the length of the carrier.

8. The method of claim 1, wherein the carrier and the dispensing member are simultaneously moving towards or away from one another.

9. The method of claim 1, wherein said skin conditioning composition is heated to at least the melting point of said skin conditioning composition to form a liquid during the application step.

10. The method of claim 1, further comprising a step of forming said carrier selected from the group consisting of molding said carrier and extruding said carrier.

11. The method of claim 1, wherein the method is performed continuously.

12. The method of claim 1, wherein said step of dispensing said skin conditioning composition comprises a step of slot coating said fluid composition into or onto said receiving region.

13. The method of claim 12, wherein a slot die coater is used during slot coating.

14. The method of claim 1, wherein said step of dispensing said skin conditioning composition comprise a step of nozzle coating said fluid composition into or onto said receiving region.

15. The method of claim 14, wherein said step of nozzle coating uses a melt-pump and a dispensing nozzle.

16. The method of claim 1, further comprising a step of dispensing a second layer of a skin conditioning composition adjacent to said skin conditioning composition present in said receiving region.

17. (canceled)

18. (canceled)

19. (canceled)

20. (canceled)
